(19) United States
(12) Patent Application Publication
(10) Pub. No.: US 2018/0325468 A1
(43) Pub. Date: Nov. 15, 2018

(54) MONITORING DEVICE WITH MULTI-PARAMETER HYPERVENTILATION ALERT

(71) Applicant: Eric Helfenbein, Sunnyvale, CA (US)

(72) Inventor: Eric Helfenbein, Sunnyvale, CA (US)

(21) Appl. No.: 15/755,811

(22) PCT Filed: Aug. 22, 2016
(86) PCT No.: PCT/IB2016/054996

§ 371 Date: Feb. 27, 2018

Related U.S. Application Data
(60) Provisional application No. 62/211,925, filed on Aug. 31, 2015.

(51) Int. Cl. A61B 5/00; A61B 5/08; A61B 5/083; A61M 16/00

(52) U.S. Cl. CPC A61B 5/746; A61B 5/0816; A61B 5/0836; A61M 16/0051; A61N 1/3904; A61M 2205/18; A61M 2230/432; A61M 2205/054; A61B 5/7282

(57) ABSTRACT

A monitoring device having a capnography capability employs a ventilation monitoring controller including a capnography monitor and a respiration monitor (21) for determining between a non-hyper-ventilating ventilation being applied to a patient and a hyperventilating ventilation being applied to the patient. In operation, the capnography monitor (20) analyzes a capnography waveform of the patient. The respiration monitor (21) determines the non-hyperventilating ventilation being applied to the patient based on an indication by an end-tidal carbon dioxide expired by the patient and/or a respiratory rate of the patient derived, partially or entirely, from the analysis of the capnography waveform by the capnography monitor (20), and determines the hyperventilating ventilation being applied to the patient based on a collective indication by both the end-tidal carbon dioxide expired by the patient and the respiratory rate of the patient derived, partially or entirely, from the analysis of the capnography waveform by the capnography monitor (20).

### FIELD OF THE INVENTION

The present disclosure generally relates to a determination of a hyperventilating ventilation being applied to a patient needing forced ventilation due to airway problems, failure to ventilate, failure to oxygenate or any other reason. The present disclosure more particularly relates to a multi-parameter alert of a determined of a hyperventilating ventilation being applied to a patient based on a level of end-tidal carbon dioxide expired by the patient and a respiratory rate of the patient.

### BACKGROUND OF THE INVENTION

In cardiac arrest (CA) resuscitation, inadvertent hyperventilation of intubated patients by Advanced Life Support (ALS) rescuer paramedics is dangerous, and may decrease the probability of a successful resuscitation. For patients with traumatic brain injury (TBI), an inadvertent hyperventilation is also associated with poor outcomes.

More particularly, during CA resuscitation or TBI treatment, an unconscious patient is often intubated with an endotrachial tube or other type of advanced airway, and is manually ventilated by a rescuer paramedic typically using an ambu-bag or a bag-valve mask combination to provide air to the lungs. While manually ventilating the patient, the rescuer paramedic must be cautious to avoid ventilating the patient at too high a rate as this could have serious repercussions on survivability and outcome. End-tidal carbon dioxide (etCO2) is the highest point on the end of each exhalation's CO2 plateau, and is representative of gas exchange in the lungs. Numerous studies have shown that the rescuer paramedic may tend to inadvertently hyperventilate the patient, causing the etCO2 to drop to too low a level indicating poor gas exchange in the lungs of the patient.

Capnography, a known monitoring of expired carbon dioxide (CO2), has been recommended in American Heart Association guidelines and has become the standard of care during resuscitations. Advanced Life Support (ALS) defibrillator/monitors have options for CO2 monitoring where a filter line (i.e., small tube) is placed in the airway circuit and gas is sucked by a small pump into a sensor (side-stream technique), or a sensor itself is placed in the airway circuit (mainstream technique). Both types of sensors are used to produce a CO2 waveform. This is usually accompanied by a capnography monitor that analyzes the resulting CO2 waveform and generates a capnography waveform for computing an etCO2 and a respiration rate. The capnography monitor usually has a capability to generate a single-parameter alert/alarm when the computed etCO2 is below an end-tidal carbon dioxide threshold or alternatively, a single-parameter alert/alarm when the computed respiration rate is above a respiration rate threshold. A problem is that a single parameter alert on etCO2 or respiration rate often creates a false alarm, or does not trigger when there is a need to modify care to address hyperventilation of the patient.

More particularly, a recommended target range of etCO2 can be quite different for a consciously breathing patient compared to an unconscious patient receiving cardiopulmonary resuscitation (CPR) and manual ventilation. Likewise, a conscious patient recovering from a cardiac arrest or trauma may by breathing at a high rate (e.g., panting), but with shallow breaths with low tidal volume, and thus have adequate gas exchange and acceptable etCO2. This is very different from a rescuer paramedic working on an unconscious patient and is inadvertently hyperventilating the patient by bagging at a high rate with full tidal volume and thus driving etCO2 dangerously low. For these reasons, the single parameter etCO2 alarm limit and the single parameter ventilation rate alarm limit are often set wider than the alarm limits should be for a given situation or disabled altogether. This sets up situations in which the rescuer paramedic may be inadvertently hyperventilating the patient and unaware of this fact.

There currently exist visual or audio metronomes that flash or beep at a fixed rate to be used for timing of manual ventilations. However, these metronomes have no feedback or sensor mechanisms and are unaware of the etCO2 levels. Also, different ventilation rates may be appropriate under different situations.

### SUMMARY OF THE INVENTION

The present disclosure provides inventions providing monitoring devices having capnography capability employing a ventilation monitoring controller including a respiration monitor for a multi-parameter hyperventilation alert when a respiration rate of a patient and an end-tidal CO2 expired by the patient collectively indicate a hyperventilating ventilation is being applied to the patient, particularly by an Advanced Life Support (ALS) rescuer. The multi-parameter hyperventilation alert of the present disclosure was vigorously tested to support a hypothesis that the multi-parameter hyperventilation alert of the present disclosure was an improvement over the single-parameter and independent etCO2 and ventilation rate alerts.

One form of the inventions of the present disclosure is a monitoring device having capnography capability (e.g., capnography, ECG or defibrillation) employs a ventilation monitoring controller including a capnography monitor and a respiration monitor for determining between a non-hyperventilating ventilation being applied to a patient and a hyperventilating ventilation being applied to the patient. In operation, the capnography monitor analyzes a capnography waveform of the patient. The respiration monitor determines the non-hyperventilating ventilation being applied to the patient based on an indication by an end-tidal carbon dioxide expired by the patient and/or a respiratory rate of the patient derived, partially or entirely, from the analysis of the capnography waveform by the capnography monitor, and determines the hyperventilating ventilation being applied to the patient based on a collective indication by both the end-tidal carbon dioxide expired by the patient and the respiratory rate of the patient derived, partially or entirely, from the analysis of the capnography waveform by the capnography monitor.

The respiration monitor may monitor the end-tidal carbon dioxide relative to an end-tidal carbon dioxide threshold delineating the non-hyperventilating ventilation being applied to a patient and the hyperventilating ventilation being applied to the patient, and/or the respiration monitor may monitor the respiratory rate of the patient relative to a respiration rate threshold delineating the non-hyperventilation respiration by the patient and the hyperventilation by the patient.

For purposes of the inventions of the present disclosure, terms of the art including, but not limited to, “monitoring device”, “ventilation”, “capnography”, “end-tidal CO2” and “respiration” are to be interpreted as understood in the art of the present disclosure and as exemplary described herein.

More particularly, the term “ventilation” broadly encompasses any and all actions executed for providing air to a patient, particularly actions executed by a ALS rescuer paramedic, the term “non-hyperventilating ventilation” broadly encompasses an execution of such ventilation actions in a manner that leads away from an inadvertent hyperventilation by the patient, and the term “hyperventilating ventilation” broadly encompasses an execution of such ventilation actions in a manner that leads to an inadvertent hyperventilation by the patient.

For purposes of the inventions of the present disclosure, the term “controller” broadly encompasses all structural configurations of an application specific main board or an application specific integrated circuit housed within or linked to a monitoring device for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s).

Examples of monitoring devices having capnography capability include, but are not limited to, a carbon dioxide monitoring device (e.g., Capnography Extension), a bed-side monitoring ECG device (e.g., IntelliVue monitors, SureSigns monitors, and Goldway monitors) and advanced life support monitoring products (e.g., HeartStart MRx and HeartStart XL defibrillators, and Efficia DFM100 defibrillator/monitor).

For purposes of the inventions of the present disclosure, the term “application module” broadly encompasses a component of the controller consisting of an electronic circuit and/or an executable program (e.g., executable software and/firmware) for executing a specific application.

For purposes of the inventions of the present disclosure, descriptive labeling of a controller herein as a “ventilating monitoring” controller, an “ECG monitoring” controller and a “defibrillation” controller serves to identify a particular controller as described and claimed herein without specifying or implying any additional limitation to the term “controller”.

Similarly, for purposes of the inventions of the present disclosure, descriptive labeling of an application module herein as a “capnography monitor” module, a “respiration monitor” module, “ECG monitoring” module(s) and “defibrillation” module(s) serves to identify a particular application module as described and claimed herein without specifying or implying any additional limitation to the term “application module”.

The foregoing forms and other forms of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

FIG. 1 illustrates a flowchart representative of an exemplary embodiment of an a ventilation monitoring method in accordance with the inventive principles of the present disclosure.

FIGS. 2-4 illustrate exemplary embodiments of a ventilation monitoring controller in accordance with the inventive principles of the present disclosure.

### DETAILED DESCRIPTION OF THE PREFERRED EMBODIMENTS

To facilitate an understanding of the present disclosure, a flowchart 10 representative of a ventilation monitoring method of the present disclosure as executed by application modules in the form of a capnography monitor 20 and a respiration monitor 21 of the present disclosure will now be described herein. From this description, those having ordinary skill in the art will appreciate how to apply the inventive principles of the present disclosure to a variety of monitoring devices having a capnography capability for incorporating a multi-parameter hyperventilation alert capability based on an end-tidal CO2 expired by the patient and a respiration rate of a patient collectively indicating a hyperventilating ventilation of a patient.

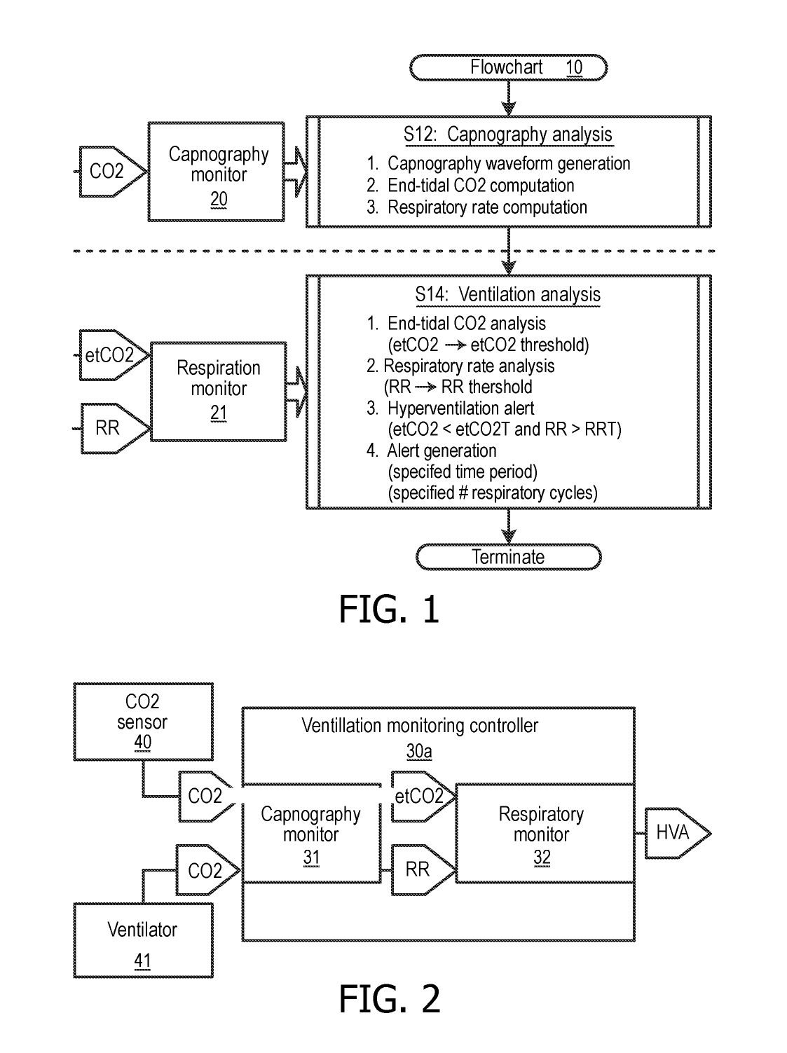

Referring to FIG. 1, a stage S12 of flowchart 10 encompasses capnography monitor 20 analyzing a capnography waveform of the patient generated from carbon dioxide expired by the patient. In practice, capnography monitor 20 may implement any technique for analyzing the capnography waveform.

In one embodiment as shown in stage S12, capnography monitor 20 generates a capnography waveform as known in the art from received carbon dioxide CO data informative of the carbon dioxide expired by the patient. From the generated capnography waveform, capnography monitor 20 computes end-tidal CO2 expired by the patient and a respiration rate of the patient as known in the art.

A stage S14 of flowchart 10 encompasses respiration monitor 21 analyzing a ventilation being applied to the patient based on the end-tidal carbon dioxide expired by the patient and the respiratory rate of the patient derived from the capnography analysis performed by the capnography monitor 20. The ventilation analysis involves the respiration monitor 21 determining a non-hyperventilating ventilation being applied to the patient based on an indication by the end-tidal carbon dioxide expired by the patient and/or the respiratory rate of the patient derived, partially or entirely, from an analysis of the capnography waveform by the capnography monitor 20. Conversely, the ventilation analysis involves the respiration monitor 21 determining a hyperventilating ventilation being applied to the patient based on a collective indication by both the end-tidal carbon dioxide expired by the patient and the respiratory rate of the patient derived, partially or entirely, from an analysis of the capnography waveform by the capnography monitor 20.

In practice, respiration monitor 21 may implement any technique for analyzing the ventilation being applied to the patient based on the end-tidal carbon dioxide expired by the patient and the respiratory rate of the patient derived from the capnography analysis performed by the capnography monitor 20.

In one embodiment as shown in stage S14, respiration monitor 21 monitors the end-tidal CO2 as computed by capnography monitor 20 relative to an end-tidal carbon dioxide threshold delineating a non-hyperventilating ventilation being applied to the patient and a hyperventilating ventilation being applied to the patient. In practice, the end-tidal carbon dioxide threshold may be empirically determined and/or set by an operator of the monitoring device. For example, the present disclosure has empirically determined a preferably 25 mmhg for the end-tidal carbon dioxide threshold.

Respiration monitor 21 also monitors the respiratory rate as computed by capnography monitor 20 relative to a respiratory threshold delineating a non-hyperventilating ventilation being applied to the patient and a hyperventilating ventilation being applied to the patient. In practice, the respiration rate threshold also may be empirically determined and/or set by an operator of the monitoring device. For example, the present disclosure has empirically determined a preferable 12 breaths per minute (bpm) for the respiration rate threshold.

From the threshold monitoring, in practice, respiration monitor 21 may implement any technique for detecting the end-tidal carbon dioxide expired by the patient being greater than (or equal to) the end-tidal carbon dioxide threshold (e.g., >25 mmhg or ≥25 mmhg) AND/OR the respiratory rate of the patient being less than (or equal to) the respiratory threshold (e.g., <12 bpm or ≤12 bpm), and for detecting the end-tidal carbon dioxide expired by the patient being less than (or equal to) the end-tidal carbon dioxide threshold (e.g., <25 mmhg or ≤25 mmhg) AND the respiratory rate of the patient being greater than (or equal to) the respiration rate threshold (e.g., >12 bpm or ≥12 bpm).

For example, respiration monitor 21 may determine a non-hyperventilating ventilation being applied to the patient whenever respiration monitor 21 individually detects the end-tidal carbon dioxide expired by the patient is greater than (or equal to) the end-tidal carbon dioxide threshold AND/OR the respiratory rate of the patient is less than (or equal to) the respiratory threshold.

Conversely, respiration monitor 21 may determine a hyperventilating ventilation being applied to the patient whenever respiration monitor 21 concurrently detects the end-tidal carbon dioxide expired by the patient is less than (or equal to) the end-tidal carbon dioxide threshold AND the respiratory rate of the patient is greater than (or equal to) the respiration rate threshold.

Also by example, respiration monitor 21 may determine a non-hyperventilating ventilation being applied to the patient whenever respiration monitor 21 individually detects the end-tidal carbon dioxide expired by the patient is less than (or equal to) the end-tidal carbon dioxide threshold AND/OR the respiratory rate of the patient is greater than (or equal to) the respiratory threshold for a duration less than a specified time period or a specified number of respiration cycles.

Conversely, respiration monitor 21 may determine a hyperventilating ventilation being applied to the patient whenever respiration monitor 21 concurrently detects the end-tidal carbon dioxide expired by the patient is less than (or equal to) the end-tidal carbon dioxide threshold AND the respiratory rate of the patient is greater than (or equal to) the respiration rate threshold for a duration greater than the specified time period or the specified number of respiration cycles.

In practice, the specified time period and the specified number of respiration cycles may be empirically determined and/or set by an operator of the monitoring device. For example, the present disclosure has empirically determined a preferable fifteen (15) seconds for the specified time period and three (3) respiration cycles for the specified number of respiration cycles. Another approach is that the etCO2 value and the respiration rate value are calculated by averaging or smoothing (using any number of techniques known to those in the field, such as median filtering the instantaneous values) over a specified time period or number of respiration cycles before comparison to thresholds, which also may be empirically determined and/or set by an operator of the monitoring device. In addition, a “hysteresis” filter may be applied to the detection of hyperventilation events, counting up towards creating of a hyperventilation alert, and conversely counting down towards the determination that the hyperventilation condition has ended. Any or all of the above methods can be applied to the device to optimize both hyperventilation condition detection sensitivity (detection of true events) and specificity (rejection of false alarms), the tradeoffs and optimization methods well known to those in the field.

Upon a determination by respiration monitor 21 of a hyperventilating ventilation being applied to the patient, respiration monitor 21 generates a hyperventilation alert in any suitable form, particularly a visual message and/or an audible alarm (e.g. “HYPERVENTILATION: HIGH RESPIRATION RATE/LOW etCO2 DETECTED”). In practice, respiration monitor 21 generates the hyperventilation alert during any determination of a hyperventilating ventilation being applied to the patient, and may extend the hyperventilation alert for a specified time period or a specified number of respiratory cycles upon a subsequent determination of a non-hyperventilating ventilation being applied to the patient. Also in practice, respiration monitor 21 may control communication of the hyperventilation alert to an operator/monitor of the monitoring device, particularly a display or a broadcast of the hyperventilation alert, or communicate the hyperventilation alert to another device for communication to an operator/monitor of the other device.

In summary, flowchart 10 represents a ventilation monitoring method executed by capnography monitor 20 and respiration monitor 21 for minimizing inadvertent hyperventilation of a patient during an emergency ventilation procedure including, but not limited to, a cardiac arrest (CA) resuscitation of a patient, a post-traumatic injury to a patient (e.g., brain injury, rib fractures, inhalation of a foreign object, bronchospasm, etc.), an overdosed patient (e.g., opioids), a patient experiencing acute laryngeal edema (e.g. inhalation burn, Ludwig's angina, epiglottitis), a patient experiencing neurological problems (e.g., sedation, narcosis, stroke, spinal cord injury, cervical—loss of diaphragmatic function, thoracic—loss of intercostal, nerve injury).

After initiation, flowchart 10 is continually executed by capnography monitor 20 and respiration monitor 21 until such time flowchart 10 is terminated by an operator/monitor of the monitoring device.

To facilitate further understanding of the present disclosure, FIGS. 2-4 illustrate ventilation monitoring controllers for a capnography monitor and a respiration monitor of the present disclosure. From a description of FIGS. 2-4, those having ordinary skill in the art will appreciate how to employ ventilation monitoring controllers for a capnography monitor and a respiration monitor of the present disclosure within any monitoring device having capnography capability.

Referring to FIG. 2, a ventilation monitoring controller 30a includes a capnography monitor 31 and a respiration monitor 32 of the present disclosure. For this controller embodiment, ventilation monitoring controller 30a is connected to any type of CO2 sensor 40 as known in the art and/or any type of ventilator 41 as known in the art for purposes an execution by capnography monitor 31 and respiration monitor 32 of a ventilation monitoring method of the present disclosure as exemplary shown in FIG. 1. Additionally, ventilation monitoring controller 30a is connected to another device (not shown) (e.g., an ECG monitoring device) for communicating any hyperventilation alert HVA from respiration monitor 32 to that device.

In practice, ventilation monitoring controller 30 may be incorporated within any type of carbon dioxide monitoring device including, but not limited to, a Capnography Extension commercially distributed by Philips Medical Systems.

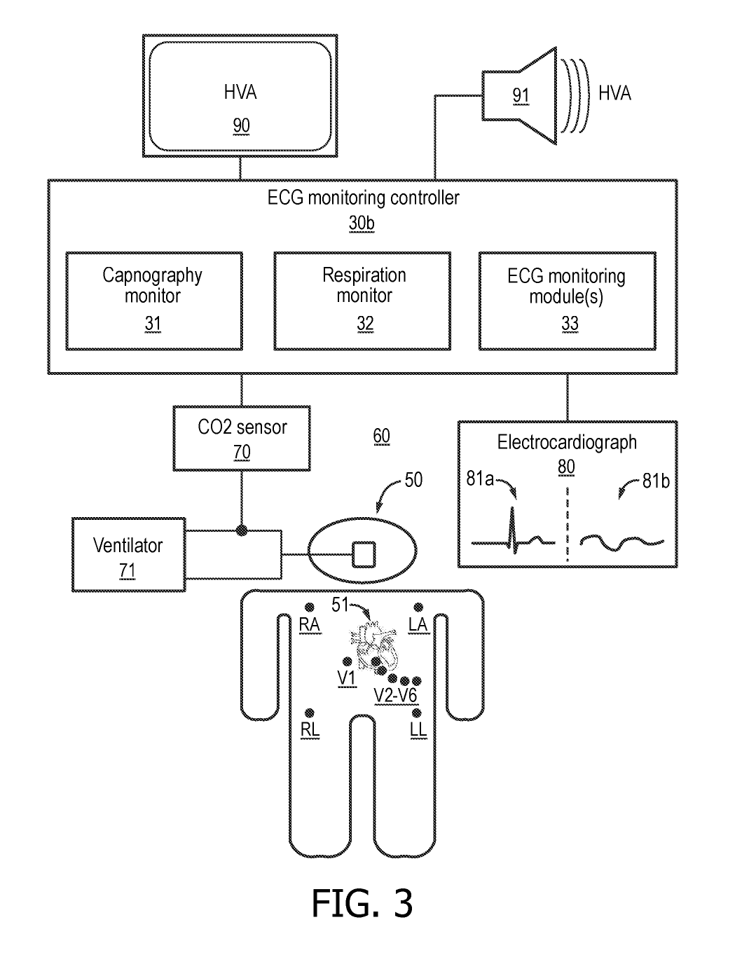

Referring to FIG. 3, an ECG monitoring device 60 employs an ECG monitoring controller 30b, an electrocardiograph 80, a display monitor 90 and a speaker 91.

ECG monitoring controller 30b includes capnography monitor 31 and respiration monitor 32 of the present disclosure. For this controller embodiment, ECG monitoring controller 30b is connected to any type of CO2 sensor 70 as known in the art and/or any type of ventilator 71 as known in the art for purposes of an execution by capnography monitor 31 and respiration monitor 32 of a ventilation monitoring method of the present disclosure as exemplary shown in FIG. 1. Additionally, ECG monitoring controller 30b is connected to display monitor 90 and speaker 91 for respectively displaying and broadcasting any hyperventilation alert HVA from respiration monitor 32.

Electrocardiograph 80 is structurally configured as known in the art to process ECG leads RA, LA, RL, LL and V1-V6 attached to a surface of a patient 50 for measuring and recording an electrocardiogram 81 of heart 51 of patient 50. Electrocardiograph 80 employs a digital signal processor (not shown) for streaming processed ECG leads to ECG monitoring controller 30b whereby ECG monitoring controller 30b additionally includes ECG monitoring module(s) 33 as known in the art for displaying and analyzing an ECG waveform 81 of any form including, but not limited to, an organized heartbeat 81a and an unorganized heartbeat 81b.

In practice ECG monitoring device 60 may be any type of ECG monitoring device having capnography capability including, but not limited to, bed-side monitoring ECG device (e.g., IntelliVue monitors, SureSigns monitors, and Goldway monitors).

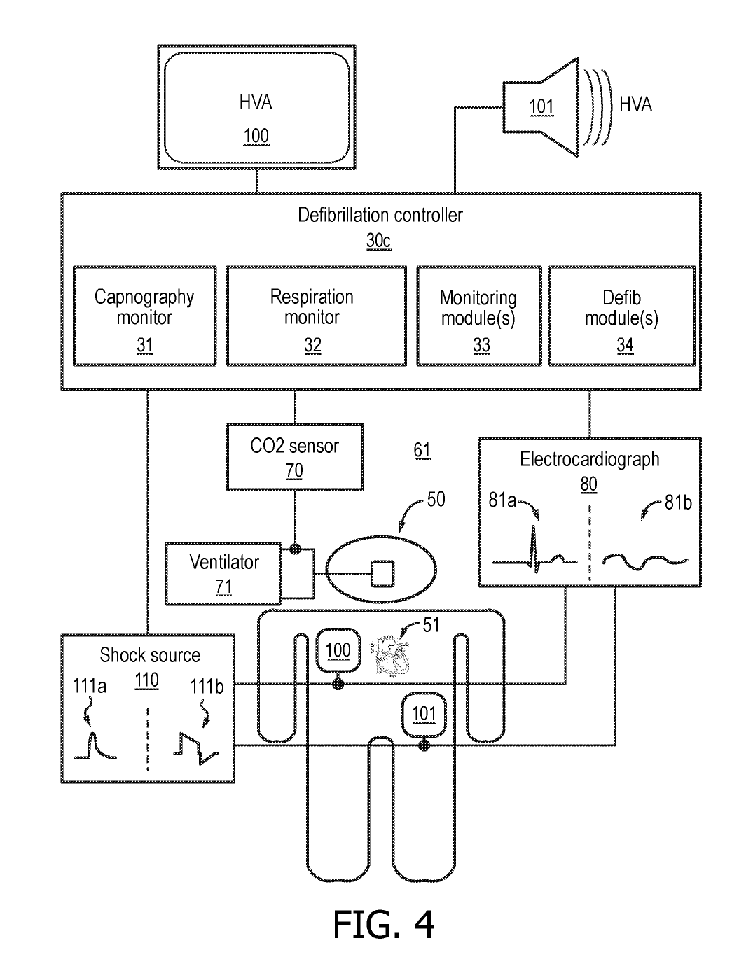

Referring to FIG. 4, a defibrillation monitoring device 60 employs ECG monitoring device 60 (FIG. 3) and a shock source 110 employing a high voltage capacitor bank (not shown) for storing a high voltage via a high voltage charger and a power supply upon a pressing of a charge button (not shown). Shock source 110 further employs a switching/isolation circuit (not shown) for selectively applying a specific waveform of an electric energy charge from the high voltage capacitor bank to electrode pads 100 and 101. Examples of the waveform include, but are not limited to, a monophasic sinusoidal waveform (positive sine wave) 111a and a biphasic truncated waveform 111b.

Electrode pads 100 and 101 are structurally configured as known in the art to be conductively applied to a patient 50 in an anterior-apex arrangement as shown in FIG. 4 or in an anterior-posterior arrangement (not shown). Electrode pads 100 and 101 conduct a defibrillation shock from shock source 110 to a heart 51 of patient 50, and are connected to electrocardiograph 80 to conduct electrical activity of heart 51 of patient 50 to electrocardiograph 80.

Defibrillation controller 30c incorporates ECG monitoring controller 30b (FIG. 3) and additionally includes defibrillation module(s) 34 as known in the art for controlling a defibrillation of heart 51 of patient 50 by an operator of defibrillation monitor 61.

Referring to FIGS. 3 and 4, in practice, the ECG leads and electrode pads 100 and 101 may be utilized to execute a known impedance method for computing the respiration rate of the patient. For this embodiment, a capnography monitor of the present disclosure only computes the etCO2 expired by the patient from the capnography waveform, and computes the respiratory rate via the known impedance method. Alternatively for this embodiment, a capnography monitor of the present disclosure only computes the etCO2 expired by the patient from the capnography waveform, and the respiratory rate is computed via the known impedance method by another application module of a controller of the present disclosure.

Referring to FIGS. 2-4, in practice, a flow sensor may be incorporated in addition to a CO2 sensor whereby a flow sensor would be utilized to monitor a volume of air exchanged between the patient and the ventilator to thereby detect any time when too much air is being pushed into the lungs of the patient (i.e., excess volume). Such excess volume detection may be useful for detecting a hyperventilation ventilation being applied to the patient.

Referring to FIGS. 1-4, those having ordinary skill in the art will appreciate numerous benefits of the present disclosure including, but not limited to, a minimization of inadvertent hyperventilation of patients, particularly by Advanced Life Support (ALS) rescuer paramedics.

Furthermore, as one having ordinary skill in the art will appreciate in view of the teachings provided herein, features, elements, components, etc. described in the present disclosure/specification and/or depicted in the FIGS. 1-4 may be implemented in various combinations of electronic components/circuitry, hardware, executable software and executable firmware and provide functions which may be combined in a single element or multiple elements. For example, the functions of the various features, elements, components, etc. shown/illustrated/depicted in the FIGS. 1-4 can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk-read only memory (CD-ROM), compact disk-read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of a novel and multi-parameter hyperventilation alert for any type of monitoring device employing capnography capability, it is noted that modifications and variations can be made by persons having ordinary skill in the art in light of the teachings provided herein, including the FIGS. 1-4. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. In a monitoring device having a capnography capability, a ventilation monitoring controller for determining between a non-hyperventilating ventilation being applied to a patient and a hyperventilating ventilation being applied to the patient, the ventilation monitoring controller comprising:
a capnography monitor operable to analyze a capnography waveform of a patient; and
a respiration monitor,
wherein the respiration monitor is operable to determine the non-hyperventilating ventilation being applied to the patient based on an indication by at least one of an end-tidal carbon dioxide expired by the patient and a respiratory rate of the patient derived from at least the analysis of the capnography waveform by the capnography monitor (20), and
wherein the respiration monitor is operable to determine a hyperventilating ventilation being applied to the patient based on a collective indication by both the end-tidal carbon dioxide expired by the patient and the respiratory rate of the patient derived from the at least the analysis of the capnography waveform by the capnography monitor;
wherein the respiration monitor is operable to generate a hyperventilation alert responsive to a determination by the respiration monitor of a hyperventilating ventilation being applied to the patient; and
a connection with another device, wherein the ventilation monitoring controller communicates the hyperventilation alert from the respiration monitor to said another device.

2. The ventilation monitoring controller of claim 1,
wherein the analysis of the capnography waveform by the capnography monitor includes:
the capnography monitor being operable to compute the end-tidal carbon dioxide expired by the patient; and
where a determination by the respiration monitor between the non-hyperventilating ventilation being applied to the patient and the hyperventilating ventilation being applied to the patient includes:
the respiration monitor being operable to monitor the end-tidal carbon dioxide as computed by the capnography monitor relative to an end-tidal carbon dioxide threshold delineating the non-hyperventilating ventilation being applied to the patient and the hyperventilating ventilation being applied to the patient.

3. The ventilation monitoring controller of claim 2,
wherein a determination by the respiration monitor of the non-hyperventilating ventilation being applied to the patient includes:
the respiration monitor being operable to detect the end-tidal carbon dioxide as computed by the capnography monitor being greater than the end-tidal carbon dioxide threshold; and
wherein a determination by the respiration monitor of the hyperventilating ventilation being applied to the patient includes:
the respiration monitor being operable to detect the end-tidal carbon dioxide as computed by the capnography monitor being less than the end-tidal carbon dioxide threshold.

4. The ventilation monitoring controller of claim 2,
wherein a determination by the respiration monitor of the non-hyperventilating ventilation being applied to the patient includes:
the respiration monitor being operable to detect the end-tidal carbon dioxide as computed by the capnography monitor being less than the end-tidal carbon dioxide threshold for a duration less than a specified time period; and
wherein a determination by the respiration monitor of the hyperventilating ventilation being applied to the patient includes:
the respiration monitor being operable to detect the end-tidal carbon dioxide as computed by the capnography monitor being less than the end-tidal carbon dioxide threshold for a duration greater than the specified time period.

5. The ventilation monitoring controller of claim 2,
wherein a determination by the respiration monitor of the non-hyperventilating ventilation being applied to the patient includes:
the respiration monitor being operable to detect the end-tidal carbon dioxide as computed by the capnography monitor being less than the end-tidal carbon dioxide threshold for a duration less than a specified number of respiration cycles; and
wherein a determination by the respiration monitor of the hyperventilating ventilation being applied to the patient includes:
the respiration monitor being operable to detect the end-tidal carbon dioxide as computed by the capnography monitor being less than the end-tidal carbon dioxide threshold for a duration greater than the specified number of respiration cycles.

6. The ventilation monitoring controller of claim 1,
wherein an analysis of the capnography waveform by the capnography monitor includes:
the capnography monitor being operable to compute the respiratory rate of the patient; and
where a determination by the respiration monitor between the non-hyperventilating ventilation being applied to the patient and the hyperventilating ventilation being applied to the patient includes:
the respiration monitor being operable to monitor the respiratory rate as computed by the capnography monitor relative to a respiration rate threshold delineating the non-hyperventilating ventilation being applied to the patient and the hyperventilating ventilation being applied to the patient.

7. The ventilation monitoring controller of claim 6,
wherein a determination by the respiration monitor of the non-hyperventilating ventilation being applied to the patient includes:
the respiration monitor being operable to detect the respiratory rate as computed by the capnography monitor being less than the respiratory rate threshold; and
wherein a determination by the respiration monitor -of the hyperventilating ventilation being applied to the patient includes:
the respiration monitor being operable to detect the respiratory rate as computed by the capnography monitor being greater than the respiratory rate threshold.

8. The ventilation monitoring controller of claim 6,
wherein a determination by the respiration monitor of the non-hyperventilating ventilation being applied to the patient includes:
the respiration monitor being operable to detect the respiratory rate as computed by the capnography monitor being greater than the respiratory rate threshold for a duration less than a specified time period; and
wherein a determination by the respiration monitor of the hyperventilating ventilation being applied to the patient includes:
the respiration monitor being operable to detect the respiratory rate as computed by the capnography monitor being greater than the respiratory rate threshold for a duration greater than the specified time period.

9. The ventilation monitoring controller of claim 6,
wherein a determination by the respiration monitor of the non-hyperventilating ventilation being applied to the patient includes:
the respiration monitor being operable to detect the respiratory rate as computed by the capnography monitor being greater than the respiratory rate threshold for a duration greater than a specified number of respiration cycles; and
wherein a determination by the respiration monitor of the hyperventilating ventilation being applied to the patient includes:
the respiration monitor being operable to detect the respiratory rate as computed by the capnography monitor being greater than the respiratory rate threshold for a duration greater than the specified number of respiration cycles.

10. The ventilation monitoring controller of claim 1,
wherein an analysis of the capnography waveform by the capnography monitor includes:
the capnography monitor being operable to compute the end-tidal carbon dioxide expired by the patient and to compute the respiration rate of the patient; and
where a determination by the respiration monitor between the non-hyperventilating ventilation being applied to the patient and the hyperventilating ventilation being applied to the patient includes:
the respiration monitor being operable to monitor the end-tidal carbon dioxide as computed by the capnography monitor (relative to an end-tidal carbon dioxide threshold delineating the non-hyperventilating ventilation being applied to the patient and the hyperventilating ventilation being applied to the patient; and
the respiration monitor being further operable to monitor the respiratory rate as computed by the capnography monitor relative to a respiration rate threshold delineating the non-hyperventilating ventilation being applied to the patient and the hyperventilating ventilation being applied to the patient.

11. The ventilation monitoring controller of claim 10,
wherein a determination by the respiration monitor of the non-hyperventilating ventilation being applied to the patient includes:
the respiration monitor being operable to detect an individual occurrence of at least one of
the end-tidal carbon dioxide as computed by the capnography monitor being greater than the end-tidal carbon dioxide threshold, and
the respiratory rate as computed by the capnography monitor being less than the respiratory rate threshold; and
wherein a determination by the respiration monitor of the hyperventilating ventilation being applied to the patient includes:
the respiration monitor being operable to detect a concurrent occurrence of
the end-tidal carbon dioxide as computed by the capnography monitor being less than the end-tidal carbon dioxide threshold, and
the respiratory rate as computed by the capnography monitor being greater than the respiratory rate threshold.

12. The ventilation monitoring controller of claim 10,
wherein a determination by the respiration monitor of the non-hyperventilating ventilation being applied to the patient includes:
the respiration monitor being operable to detect an individual occurrence of at least one of
the end-tidal carbon dioxide as computed by the capnography monitor being less than the end-tidal carbon dioxide threshold for a duration less than a specified time period, and
the respiratory rate as computed by the capnography monitor being greater than the respiratory rate threshold for the duration less than the specified time period; and
wherein a determination by the respiration monitor of the hyperventilating ventilation being applied to the patient includes:
the respiration monitor being operable to detect a concurrent occurrence of
the end-tidal carbon dioxide as computed by the capnography monitor being less than the end-tidal carbon dioxide threshold for a duration greater than the specified time period, and
the respiratory rate as computed by the capnography monitor being greater than the respiratory rate threshold for the duration greater than the specified time period.

13. The ventilation monitoring controller of claim 10,
wherein a determination by the respiration monitor of the non-hyperventilating ventilation being applied to the patient includes:
the respiration monitor being operable to detect an individual occurrence of at least one of
the end-tidal carbon dioxide as computed by the capnography monitor being less than the end-tidal carbon dioxide threshold for a duration less than the specified number of respiration cycles, and
the respiratory rate as computed by the capnography monitor being greater than the respiratory rate threshold for the duration less than the specified number of respiration cycles; and
wherein a determination by the respiration monitor the hyperventilating ventilation being applied to the patient includes:
the respiration monitor being operable to detect a concurrent occurrence of
the end-tidal carbon dioxide as computed by the capnography monitor being less than the end-tidal carbon dioxide threshold for a duration greater than the specified number of respiration cycles, and
the respiratory rate as computed by the capnography monitor being greater than the respiratory rate threshold for the duration greater than the specified number of respiration cycles.

14. The ventilation monitoring controller of claim 11,
wherein the hyperventilation alert is at least one of a visual message and an audible alarm; and
wherein the respiration monitor is operable to terminate the hyperventilation alert responsive to a subsequent determination by the respiration monitor of the non-hyperventilating ventilation being applied to the patient.

15. The ventilation monitoring controller of claim 1, wherein the monitoring device is one of a carbon dioxide monitoring device, an electrocardiogram monitoring device, and a defibrillation monitoring device.

16. In a monitoring device incorporating a ventilation monitoring controller including a capnography monitor and a respiration monitor, a ventilation monitoring method for determining between a non-hyperventilating ventilation being applied to a patient and a hyperventilating ventilation being applied to the patient, the ventilation monitoring method comprising:
the capnography monitor analyzing a capnography waveform of a patient;
the respiration monitor determining the non-hyperventilating ventilation being applied to the patient based on an indication by at least one of an end-tidal carbon dioxide expired by the patient and a respiratory rate of the patient derived from at least the analysis of the capnography waveform by the capnography monitor; and
the respiration monitor determining a hyperventilating ventilation being applied to the patient based on a collective indication by both the end-tidal carbon dioxide expired by the patient and the respiratory rate of the patient derived from the at least the analysis of the capnography waveform by the capnography monitor; and
the ventilation monitoring controller communicating with another device a hyperventilation alert, wherein the respiration monitor determines hyperventilating ventilation is being applied to the patient.

17. The ventilation monitoring method of claim 16,
wherein the analysis of the capnography waveform by the capnography monitor, includes:
the capnography monitor computing the end-tidal carbon dioxide expired by the patient and computing the respiration rate of the patient; and
where the determination by the respiration monitor between the non-hyperventilating ventilation being applied to the patient and the hyperventilating ventilation being applied to the patient includes:
the respiration monitor monitoring the end-tidal carbon dioxide as computed by the capnography monitor relative to an end-tidal carbon dioxide threshold delineating the non-hyperventilating ventilation being applied to the patient and the hyperventilating ventilation being applied to the patient; and
the respiration monitor monitoring the respiratory rate as computed by the capnography monitor relative to a respiration rate threshold delineating the non-hyperventilating ventilation being applied to the patient and the hyperventilating ventilation being applied to the patient.

18. The ventilation monitoring method of claim 17,
wherein the determination by the respiration monitor of the non-hyperventilating ventilation being applied to the patient includes:
the respiration monitor detecting an individual occurrence of at least one of
the end-tidal carbon dioxide as computed by the capnography monitor being greater than the end-tidal carbon dioxide threshold, and
the respiratory rate as computed by the capnography monitor being less than the respiratory rate threshold; and
wherein the determination by the respiration monitor of the hyperventilating ventilation being applied to the patient includes:
the respiration monitor detecting a concurrent occurrence of
the end-tidal carbon dioxide as computed by the capnography monitor being less than the end-tidal carbon dioxide threshold, and
the respiratory rate as computed by the capnography monitor being greater than the respiratory rate threshold.

19. The ventilation monitoring method of claim 17,
wherein the determination by the respiration monitor of the non-hyperventilating ventilation being applied to the patient includes:
the respiration monitor detecting an individual occurrence of at least one of
the end-tidal carbon dioxide as computed by the capnography monitor being less than the end-tidal carbon dioxide threshold for a duration less than a specified time period, and
the respiratory rate as computed by the capnography monitor being greater than the respiratory rate threshold for the duration less than the specified time period; and
wherein the determination by the respiration monitor of the hyperventilating ventilation being applied to the patient includes:
the respiration monitor detecting a concurrent occurrence of
the end-tidal carbon dioxide as computed by the capnography monitor being less than the end-tidal carbon dioxide threshold for a duration greater than the specified time period, and
the respiratory rate as computed by the capnography monitor being greater than the respiratory rate threshold for the duration greater than the specified time period.

20. The ventilation monitoring method of claim 17,
wherein the determination by the respiration monitor of the non-hyperventilating ventilation being applied to the patient includes:
the respiration monitor detecting an individual occurrence of at least one of
the end-tidal carbon dioxide as computed by the capnography monitor being less than the end-tidal carbon dioxide threshold for a duration less than the specified number of respiration cycles, and
the respiratory rate as computed by the capnography monitor being greater than the respiratory rate threshold for the duration less than the specified number of respiration cycles; and
wherein the determination by the respiration monitor of the hyperventilating ventilation being applied to the patient includes:
the respiration monitor detecting a concurrent occurrence of
the end-tidal carbon dioxide as computed by the capnography monitor being less than the end-tidal carbon dioxide threshold for a duration greater than the specified number of respiration cycles, and
the respiratory rate as computed by the capnography monitor being greater than the respiratory rate threshold for the duration greater than the specified number of respiration cycles.
